(19) United States
(12) Patent Application Publication
(10) Pub. No.: US 2009/0270680 A1
(43) Pub. Date: Oct. 29, 2009

(54) SELF-PROPELLED COLONOSCOPE

(76) Inventor: Masazumi TAKADA, Tokyo (JP)

Correspondence Address: YOUNG & THOMPSON, 209 Madison Street, Suite 500, ALEXANDRIA, VA, 22314, US

(21) Appl. No.: 12/428,653

(22) Filed: Apr. 23, 2009

(30) Foreign Application Priority Data
Apr. 25, 2008 (JP) 2008-115278

(51) Int. Cl. A61B 1/012

(52) U.S. Cl. 600/118

(57) ABSTRACT

A self-propelled colonoscope comprises a tubular flexible insertion portion (15) inserted into the colon and an endless belt (17) arranged on a circulating path on a part of the outer wall and the inner wall of the flexible insertion portion (15) The endless belt (17) travels outside the flexible insertion portion (15) initially as supported by the guide hooks (39), mounted on the outer wall of the flexible insertion portion, at the distal end portion of the flexible insertion portion (15) and then passes through the guide pipe (43) arranged on the inner wall of the flexible insertion portion (15) At least a part of the guide hook (39) is embedded in the outer wall of the flexible insertion portion (15) And, a pulley (61) is attached at the distal end of the circulating path of the endless belt (17).

### FIELD OF THE INVENTION

The present invention relates to a self-propelled colonoscope to be inserted into the colon by driving endless belts, arranged on the inner and outer walls of a flexible section of an insertion tube in a loop fashion.

### BACKGROUND OF THE INVENTION

Colonoscopic examination is currently carried out by inserting a colonoscope into the colon by hand. Since the colonoscope has to be inserted along the curves of the colon, the examination may cause the patient pain by excessively extending or bending the colon. Sometimes, the colonoscope perforates the colon wall. As a colonoscope which does not cause a patient pain, colonoscopes self-propelled along the curves of the colon have been proposed.

The inventor proposed a self-propelled colonoscope equipped with endless belts arranged on the inner and outer walls of a flexible section of the insertion tube in a loop fashion and to be inserted into the colon by driving the endless belts (Japanese Patent No. 3514252). Outside the flexible section, the endless belts are supported by guide hooks and travel in the opposite direction to the insertion direction of the colonoscope with contacting the inner wall of the colon, propelling the colonoscope into the colon. Inside the flexible section, the endless belts travel through guide pipes, mounted on the inner wall of the flexible section extending in the length direction of the flexible section. By driving the endless belts with a driving mechanism, the endless belts propel the colonoscope into the colon by friction between the endless belts and the inner wall of the colon outside the flexible section. Inside the flexible section, the endless belts travel through the guide pipes smoothly. So the colonoscope can be propelled into the colon without excessive extension and bending of the colon. Therefore, the colonoscope can be smoothly inserted into the colon while keeping the colon in relatively the same position and shape, causing the patient hardly any pain.

### SUMMARY OF THE INVENTION

The object of the invention is to provide a self-propelled colonoscope, having above described structure and function, modified so as to make a diameter of an insertable portion into the colon as small as possible and to drive the endless belts smoothly.

A self-propelled colonoscope according to the first aspect of the present invention comprises:

a tubular flexible insertion portion to be inserted into a colon;

an endless belt arranged along a circulating path on the inner wall and the outer wall of the flexible insertion portion;

a driving mechanism to drive the endless belt;

guide hooks arranged along the circulating path on the outer wall of the flexible insertion portion; and

a guide pipe extending along the circulating path on the inner wall of the flexible insertion portion and guiding the endless belt,
wherein at least a part of said guide hook is embedded in the outer surface of said flexible insertion portion

In the self-propelled colonoscope using the endless belt, since the endless belt is arranged on the outer surface of the flexible insertion portion, a radius of an insertable portion into the colon is a sum of a radius of the flexible insertion portion and a diameter of the endless belt. That is, the self-propelled colonoscope has a larger radius of the insertable portion into the colon by the diameter of the endless belt compared with the conventionally used self-propelled colonoscope. In the present invention, a part of the guide hook is embedded in the outer surface of the flexible insertion portion and the endless belt is supported by the embedded guide hook. This makes the radius of the insertable portion into the colon small by the depth of the embedded portion of the hook.

In the present invention, said flexible insertion portion may be compatible with an upper gastrointestinal endoscope in structure and size, except for said driving mechanism.

In many cases, a colonoscope has a diameter of about 14 mm and an upper gastrointestinal endoscope (gastrocamera) has a diameter of 6 mm or 9 mm. In the present invention, the flexible insertion portion is designed to have a diameter of 14 mm at a maximum and also to adjust the depth of the embedded portion of the hook whereby the insertable portion into the colon (the flexible insertion portion equipped with the endless belt) can have a diameter of about 20 mm or less (corresponding to a diameter of an anus of an adult.) A self-propelled colonoscope according to the second aspect of the present invention comprises:

a tubular flexible insertion portion to be inserted into a colon;

an endless belt arranged along a circulating path on the inner wall and the outer wall of the flexible insertion portion;

a driving mechanism to drive the endless belt;

guide hooks arranged along the circulating path on the outer wall of the flexible insertion portion; and

a guide pipe extending along the circulating path on the inner wall of the flexible insertion portion and guiding the endless belt,

wherein a pulley is arranged at the distal end of the circulating path of said endless belt.

In the self-propelled colonoscope using the endless belt according to the present invention, the endless belt travels along the circulating path on the inner wall and the outer wall of the flexible insertion portion. At the distal end of the circulating path, the endless belt turns at approximately 180°. During the turn, since the endless belt comes in contact with the inner wall of the guide pipe, frictional force between the both increases and thus it is required to drive the endless belt with large force. Accordingly, in the present invention, the pulley is arranged at the distal end of the circulating path to turn the endless belt, causing smoothly turn of the endless belt.

In the self-propelled colonoscope according to the first aspect of the present invention, a pulley may be arranged at the distal end of the circulating path of said endless belt.

In the present invention, a rotating axis for supporting said pulley rotatably may be supported to said guide pipe.

Furthermore, in the present invention, said pulley may be arranged on the side wall of said guide hole, at which said guide pipe is opened to the outer surface of said flexible insertion portion, on the side of said driving mechanism, and

the self-propelled colonoscope further comprises:

pulley restriction means arranged at a distal side of said guide hole and opposite to said pulley; and

coupling means for coupling said pulley restriction means and the rotating axis of said pulley,

wherein said coupling means restrains said pulley to said pulley restriction means to keep it from being pulled toward said driving unit.

As described above, the endless belt is driven by the driving mechanism arranged on the side of the operation unit and also turns at approximately 180° by the pulley toward the operation unit at the distal end of the circulating path. At the turn, the pulley is subjected to force in the turn direction, that is, toward the operation unit and thus pulled toward the operation unit. In the present invention, the restriction means, arranged at a distal side of the guide hole and opposite to the pulley and also coupled to the rotating axis of the pulley, keeps the pulley from being pulled toward the operation unit.

In the present invention, the circulating path on the outer wall of the flexible insertion portion at the distal end portion of the flexible insertion portion may be 10 to 60 cm in length.

The reason for setting the length of the portion to under 60 cm is that if the endless belt has an effective driving length of about 60 cm which is a sum of a length of the transverse colon of a free segment and an allowance of 10 cm, it becomes possible to give sufficient self-propelling force to the colonoscope. “At the distal end portion of the flexible insertion portion” means “near the distal end of the flexible insertion portion”. This meaning includes a case in which a portion for 0 to 10 mm from the distal end of the insertion portion has no endless belt arranged thereon. In the present invention, since a travel distance of the endless belt which travels on the outer wall of the flexible insertion portion is set as short as possible, it makes possible to insert the flexible insertion portion into the colon with keeping flexibility.

In the present invention, said guide pipe may be made of an ultralow friction material.

In this case, since friction between the guide pipe and the endless belt can be reduced, it becomes possible to travel the endless belt more smoothly.

In the present invention, said pulleys may be arranged at separate positions in the length direction of said flexible insertion portion.

If the pulleys (that is, positions of the distal end of the circulating path of each endless belt, and furthermore positions of the guide holes) are arranged at the same position in the length direction of the flexible insertion portion, the diameter of the flexible insertion portion becomes large partially at the position. This increases resistance when the colonoscope is inserted. Accordingly, by arranging the pulleys at the separate positions in the length direction of the flexible insertion portion, portions increased in the diameter are dispersed in the length direction of the flexible insertion portion, causing smooth insertion of the flexible insertion portion.

### EFFECT OF THE INVENTION

As described above, according to the present invention, since a part of the guide hook which supports the endless belt is embedded in the flexible insertion portion, a diameter of an insertable portion into the colon can make as small as possible. And, a pulley is arranged at the distal end of the circulating path of the endless belt so as to turn the endless belt, causing smooth turn and stable travel of the endless belt.

### BRIEF DESCRIPTION OF THE DRAWINGS

FIG. 1 is a perspective drawing showing the self-propelled colonoscope (example) according to one design of the present invention;

FIG. 2 is a sectional drawing showing the distal section of the insertion portion of the colonoscope of FIG. 1;

FIG. 3A is a side sectional drawing showing the insertion portion and a part of the driving unit of the colonoscope of FIG. 1; FIG. 3B is an enlarged side sectional drawing showing a part of FIG. 3A; and FIG. 3C is a sectional plan drawing of FIG. 3B;

FIG. 4A is an A-A cross sectional drawing of FIG. 3A and FIG. 4A is a B-B cross sectional drawing of FIG. 3A;

FIG. 5A is a perspective drawing schematically showing the structure of the endless belt; FIG. 5B is a side drawing schematically showing the pulley on which the endless belt is wound; and FIG. 5C is a side drawing schematically showing the engagement state of the endless belt with the pulley;

FIG. 6 is a drawing schematically showing the exemplary colonoscope insertion path;

FIG. 7 are drawings showing a portion around the guide hole of the insertion tube 9, FIG. 7A is a vertical section drawing and FIG. 7B is a plan drawing;

FIG. 8 are transverse section drawings showing a portion around the guide hole of the insertion tube; and

FIG. 9 is a drawing schematically showing a portion around the guide hole of the insertion tube of the self-propelled colonoscope according to another embodiment of the present invention.

### DESCRIPTION OF THE PREFERRED EMBODIMENT

Referring to the attached drawings, the details of the design of the present invention will be presented.

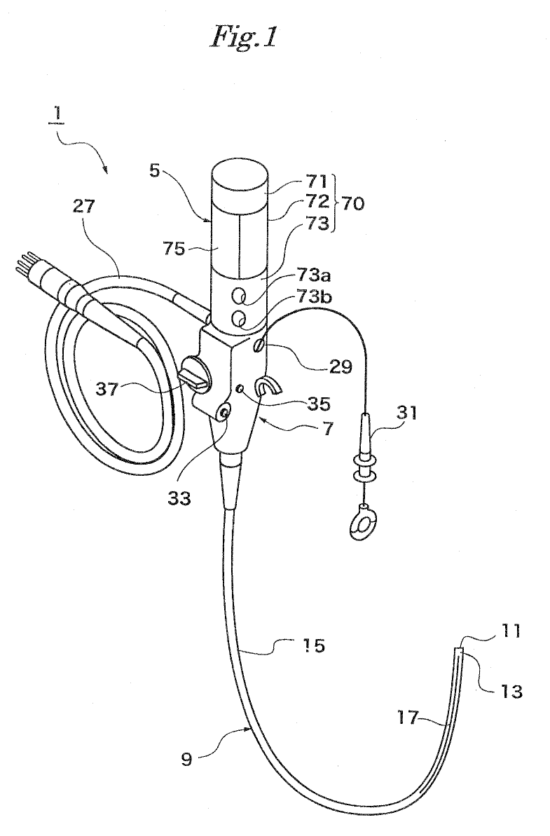

FIG. 1 is a perspective drawing showing the self propelled colonoscope (example) according to one design of the present invention.

A self-propelled colonoscope 1 is provided with a belt driving unit 5 covered with a driving unit casing 70, an operation unit 7 under the belt driving unit 5, an insertion portion (insertion tube) 9 extending from the operation unit 7 and inserted into the colon, etc. The insertion tube 9 comprises a distal section 11, a bending section 13 and a flexible section (flexible insertion portion) 15. Multiple (three, in this example) endless belts 17 are longitudinally arranged on the flexible section 15.

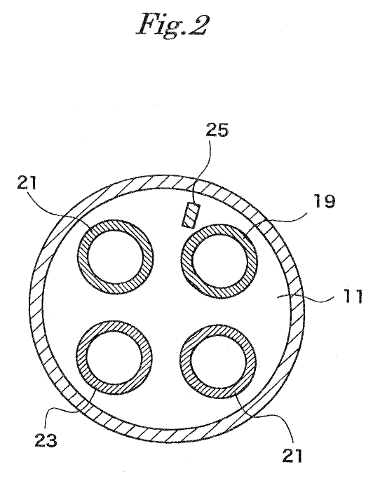

FIG. 2 is a sectional drawing showing the distal section of the insertion portion of the colonoscope of FIG. 1.

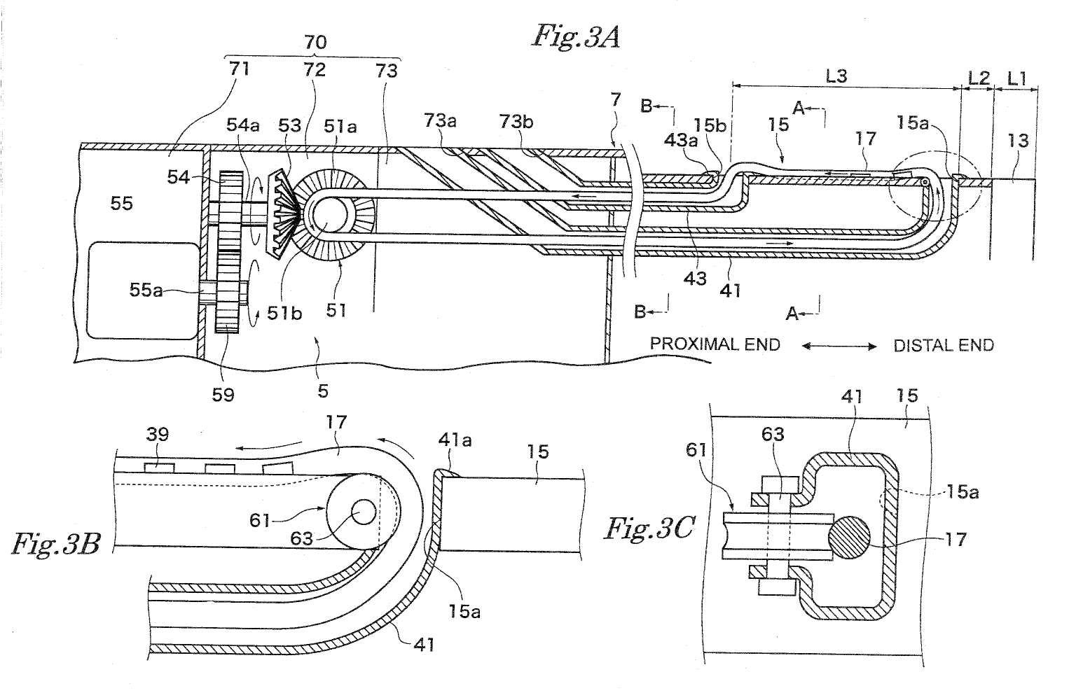

FIG. 3A is a side sectional drawing showing the insertion portion and a part of the driving unit of the colonoscope of FIG. 1; FIG. 3B is an enlarged side sectional drawing showing a part of FIG. 3A; and FIG. 3C is a sectional plan drawing of FIG. 3B.

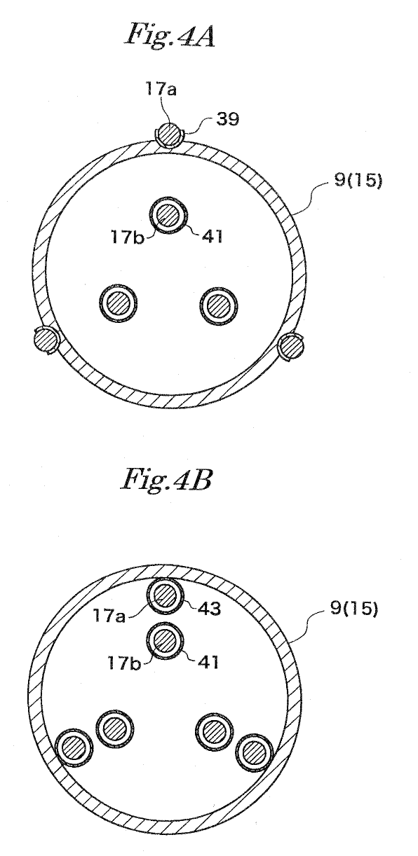

FIG. 4A is an A-A cross sectional drawing of FIG. 3A and FIG. 4A is a B-B cross sectional drawing of FIG. 3A.

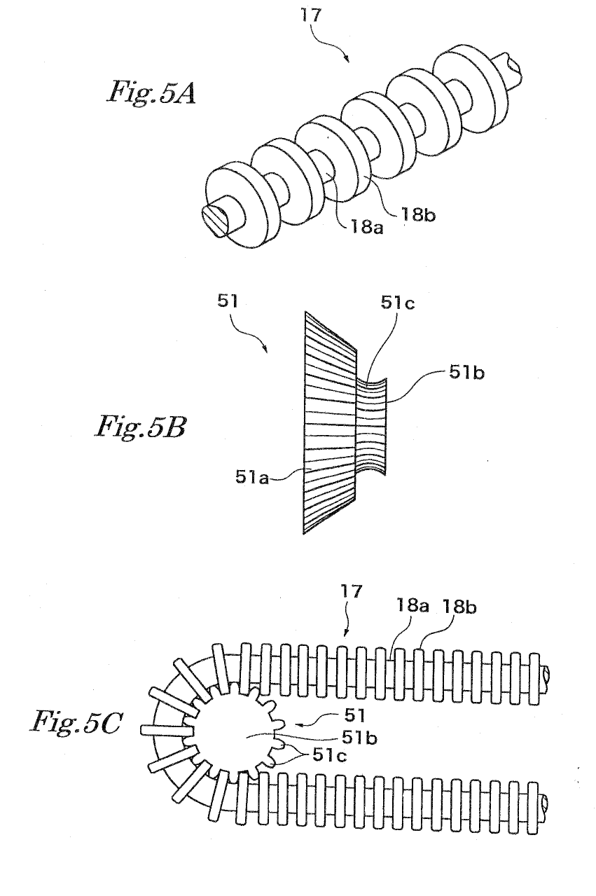

FIG. 5A is a perspective drawing schematically showing the structure of the endless belt; FIG. 5B is a side drawing schematically showing the pulley on which the endless belt is wound; and FIG. 5C is a side drawing schematically showing the engagement state of the endless belt with the pulley.

At the distal section 11 of the insertion tube 9, as shown in FIG. 2, an image receiving window 19, one or two light-projecting windows 21 (two windows in this design), a suction and forceps opening 23, and an air-water nozzle 25 are shown. The image-receiving window 19, equipped with an objective lens when the observation device is a fiberscope, or an image pick-up device, such as a CCD, when it is an electronic scope, receives an image from the distal end surface. The received image is transmitted to the operation unit 7 through the image guide of the fiberscope or the lead wire of the electronic scope, which is inserted into the insertion tube 9, and then transmitted through a universal cord 27 to a display. A light guide, such as an optical fiber, is inserted into the bore of each light-projecting window 21, runs through the operation unit 7 and is connected to an external light source via the universal cord 27. The light source projects light from the distal end surface of the light guide.

The suction and forceps opening 23 is connected to the forceps insertion opening 29 (referring to FIG. 1) in the operation unit 7, and forceps 31 is inserted therein. The working tip end of the forceps 31, protruding from the distal end of the insertion tube 9, is manipulated at the proximal part of the forceps 31 to perform procedures such as to treat a lesion or collecting tissue from a patient.

The bore of the air--water nozzle 25 is a water-air supply channel through which air or a cleaning solution flows and is injected from the air-water nozzle 25 by manipulating the air-water supply button 33 mounted on the operation unit 7. Through the suction and forceps opening 23, bodily fluid or cleaning solution remaining in the colon is sucked out and discharged to outside the patient's body. This operation is carried out by manipulating the suction control button 35 on the operation unit 7.

The flexible section 13 of the insertion tube 9 is able to bend upward and downward, rightward and leftward, and obliquely by manipulating a control knob 37 on the operation unit 7. The flexible section 13 has a length L1 (shown in FIG. 3) of about 10 cm, for example.

As shown in FIGS. 3 and 4, multiple endless belts 17 are longitudinally arranged on the flexible section 15 of the insertion tube 9. The diameter of the flexible section 15 is preferably 5 to 30 mm; within 20 mm is most preferable. In this example, three of the endless belts 17 are arranged. A larger number of endless belts 17 are preferable, because the colonoscope has greater self-propelling property as the number of endless belts increases.

As shown in FIG. 3, the endless belts 17 circulate on a circulating path on the inner wall and a part of the outer wall of the flexible section 15. The circulating path extends between the distal end of the flexible section 15 and a driving roller 51 (described later in detail) of the belt driving unit 5 stored in the driving unit casing 70 in the length direction of the flexible section 15. As described later in detail, a forward path of the circulating path on which the endless belts 17 travel toward the distal end of the flexible section 15 from the driving roller 51 is arranged on the inner wall of the flexible section 15. And, a part of a backward path of the circulating path on which the endless belts 17 travel toward the driving roller 51 from the distal end of the flexible section 15 is arranged on the outer wall of the flexible section 15 and the other part is arranged on the inner wall. Since sigmoid colon is about 42 cmm in length and transverse colon is about 45 cm in length, the backward path arranged on the outer wall of the flexible section 15 has a length of 10 to 60 cm, more preferably 20 to 60 cm.

In the forward path toward the distal end of the flexible section 15 from the driving roller 51, the endless belt 17 passes through an inner guide pipe 41 arranged on the inner wall of the flexible section 15. The inner guide pipe 41 extends between a guide pipe hole 73a formed at the driving unit casing 70 and a guide hole 15a penetrating through the wall of the distal end of the flexible section 15. As shown in FIG. 3B, the inner guide pipe 41 has a flange portion 41a at the distal end thereof. The guide pipe 41 passes through the guide hole 15a and the flange portion 41a is fixed on the outer wall. A distance L2 between the distal end of the flexible section 15 and the guide holes 15a is preferably 0 to 10 cm, as shown in FIG. 3A.

As shown in FIG. 3B, a pulley 61 is attached on the side wall of the guide hole 15a, formed on the flexible section 15, on the side of the driving unit (operation unit) (at the distal end of the circulating path of the endless belts.) As shown in FIG. 3C, both ends of a rotating axis 63 of the pulley 61 are rotatably supported to the guide pipe 41.

The endless belt 17 gets out through the guide hole 15a and turns by the pulley 61 toward the driving unit at approximately 180°. At this time, since friction is hardly caused between the endless belt 17 and the pulley 61, the endless belt 17 turns without resistance. This makes the driving force of the driving unit for the endless belts small.

In the backward path toward the driving roller 51 from the distal end of the flexible section 15, the endless belts travel on the outer wall of the flexible section 15 initially and then on the inner wall of the flexible section 15. On the outer wall of the flexible section 15, the endless belt 17 is supported by guide hooks 39 mounted on the outer wall of the flexible section 15. On the inner wall of the flexible section 15, the endless belt 17 passes through an outer guide pipe 41 mounted on the inner wall of the flexible section 15.

As shown in FIG. 3, the circulating path on the outer wall of the flexible section 15 has a length L3 of 10 to 60 cm (described later in detail) On the outer circulating path, as shown in FIG. 4A, at least a part of the guide hook 39 is embedded on the outer surface of the flexible section 15. This makes a diameter of an insertable portion into the colon small by the depth of the embedded portion of the hook, compared with the colonoscope proposed by the inventor before. The depth of the embedded portion of the hook is adjusted such that an insertable portion into the colon (the flexible section 15 on which the endless belts 17 is arranged) has a diameter of about 20 mm.

Each of the guide hooks 39 has a circular-arc cross section with a center angle over 180°, as shown in FIG. 4A. Accordingly, the outward surface of the endless belt 17 supported by the guide hooks 39 is exposed from the guide hooks 39 so as to be in contact with the inner wall of the colon with a sufficient contact area when the flexible section 15 is inserted into the colon. Even when the flexible section 15 is severely bent, the endless belts 17 will not come off the guide hooks 39.

The guide hooks 39 are mounted lengthwise along the flexible section 15 at intervals of 1 to 3 cm, in this design; however, the guide hooks 39 may be continuously placed in the lengthways direction of the flexible section.

The outer guide pipe 43 extends between a guide pipe hole 73b formed at the driving unit casing 70 and a guide hole 15b formed on a proximal side of the most proximal guide hook 39. As shown in FIG. 3A, the outer guide pipe 43 has a flange portion 43a at the distal end thereof. The guide pipe 43 passes through the guide hole 15b and the flange portion 43a is fixed on the outer wall.

As shown in FIG. 4B, the outer and inner guide pipes 43 and 41 extend parallel in the flexible section 15; however, the guide pipes 43 and 41 may be arranged diagonally as shown in FIG. 4B or circumferentially.

The outer and inner guide pipes 43 and 41 are made of an ultralow friction material (for example, ultrafine tube or ultrathin tube (manufactured by Nirei Industry Co., Ltd.). More particularly, a tube comprising an outer tube made of Pebax (registered trade name) or Hytrel (registered trade name) and an inner tube made of PEEK (registered trade name) and having an outer diameter of about 3 mm may be used. This makes friction between the guide pipes 41 and 43 and the endless belt 17 small and thus the endless belt 15 can travel through the guide pipes 41 and 43 smoothly.

As described above, the endless belt 17 gets out of the inner guide pipes 41 through the guide holes 15a at the distal end portion of the flexible section 15 and turns toward the proximal end. And then, the endless belt 17 travels outside the flexible section 15, supported by the guide hooks 39, enters the outer guide pipe 43 through the guide hole 15b and then travels toward the driving roller 51 in the opposite direction to the insertion direction.

As shown in FIG. 3, when the colonoscope is inserted, the endless belts 17, supported by the guide hooks 39 mounted on the outer wall of the flexible section 15, travel in the opposite direction to the insertion direction while remaining in contact with the inner wall of the colon outside the flexible section 15. This propels the colonoscope into the colon. When the colonoscope is removed from the patient's body, the endless belts 17 circulate in the opposite direction to the direction at the insertion. That is, the endless belts 17 travel in the insertion direction outside the flexible section 15 and travel in the opposite direction to the insertion direction inside the flexible section 15.

Each of the endless belts 17 is made of a flexible and strong material such as, for instance, carbon fiber or resin and, as shown in FIG. 5A and FIG. 5C, each comprises an axial belt 18a and multiple rack gear teeth 18b arranged lengthways along the axial belt 18a, which has a circular cross section with a diameter of 1 to 3 mm. The rack gear teeth 18b also have a circular cross section and are coaxially arranged at regular intervals on the outer surface of the axial belt 18a. The rack gear teeth 18b have a diameter of 1 to 3 mm and a thickness of 0.1 to 1.0 mm and the distance between any two of the rack gear teeth 18b is 0.1 to 1.0 mm. The diameters of the axial belt 18a and the rack gear teeth 18b are selected within these ranges so that the diameter of the rack gear teeth 18b is larger than the diameter of the axial belt 18a. The outer surface of the rack gear teeth 18b may be coated with high frictional material, and the outer surface of the pulley 51b including a pinion gear teeth 51c, as described later, may be also coated with high frictional material. The length of the endless belt 17 will be described later.

The endless belt has a circular cross section so as to be able to bend flexibly in all radial directions to the axis with equal force. So, when the insertion tube 9 is inserted along the bends of the colon, the endless belt 17 can easily follow the motion of the insertion tube 9. Since the rack gear teeth 18b are formed along the entire length of the endless belt 17, if the endless belt 17 is twisted, the rack gear teeth 18b may be in contact with the inner wall of the colon, causing the endless belt 17 to rub against it. Accordingly, the friction force between the endless belts 17 and the inner wall of the colon will be increased to improve the self-propelling ability.

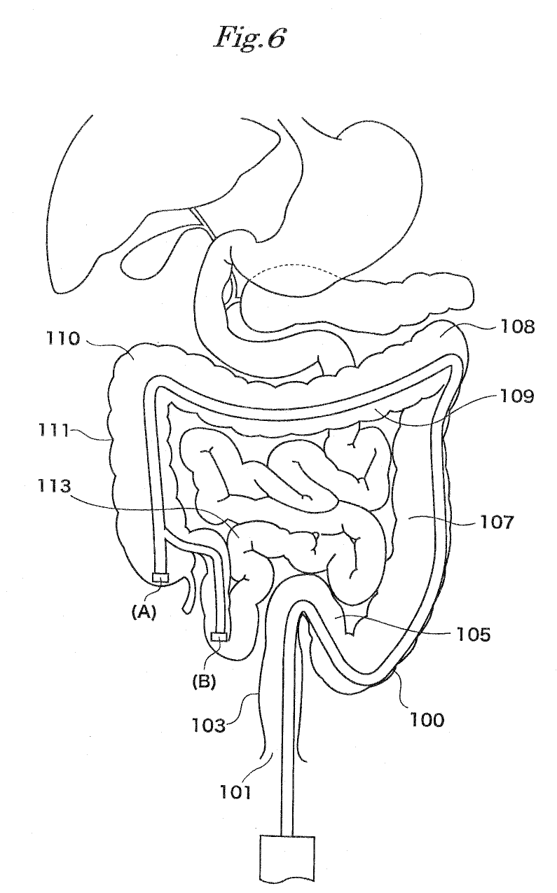

The insertion tube 9, inserted into the colon, advances from the sigmoid colon 105 to the ileum 107 through the descending colon, the transverse colon 109 and the ascending colon 111 as described later referring to FIG. 6. Since the flexible section 15 has an outer diameter as great as about 16 mm, when the colonoscope advances into the colon, the curve of the colon causes a difference in the inner ring length and the outer ring length of the inserted flexible section 15. For example, when the distal end of the insertion tube 9 reaches the ileum and the flexible section 15, having a diameter of 16 mm, turns in a curve, the outer ring length is 3.12% longer than the straight length.

Accordingly, the endless belt 17 arranged on the outer and inner walls of the flexible section 15 necessarily has an allowable length for differences in the inner and outer ring length of the flexible section 15. For this reason, the length of the endless belt 17 is designed be 102 to 104% of the length of the belt when it turns under tension from the guide hole 49 formed at the distal end of the flexible section 15 to the same guide hole 49 through the driving unit while keeping the flexible section 15 straight. Since the length of the endless belt 17 is set as above, it can sufficiently follow the bending of the flexible section 15; therefore, the colonoscope can be propelled into the colon stably.

As described above, the length of the endless belt 17 has a certain allowance; however, since the pulley 51b for driving the endless belt 17 is formed with the pinion gear teeth 51c on the circumferential surface, the endless belt 17 and the pulley 51b are tightly engaged with each other by engagement of the rack gear teeth 18b and the pinion gear teeth 51c, whereby the endless belt 17 can be driven without idling.

As described above, the guide hooks 39 are mounted only along a section between a distal position 0 to 10 cm apart from the distal end of the flexible section 15 and a proximal position 60 cm apart from the distal end of the flexible section 15. The reason will be described below.

An exemplary colonoscope insertion path will be described.

FIG. 6 is a drawing schematically showing the exemplary colonoscope insertion path.

The insertion tube of the colonoscope is inserted from the anus 101 into the rectum 103 (fixed segment) and advances from the sigmoid colon 105 (free segment) to the ileum 113 through the descending colon 107 (fixed segment), the transverse colon 109 (free segment) and the ascending colon 11.1 (fixed segment). The distal end of the insertion tube may be reached to the terminal of the colon, as shown in (A) in the figure, or to ileum 113 for about 25 cm, as shown in (B) in the figure.

In the case of the present self-propelled colonoscope, when the distal end of the insertion tube 9 enters the sigmoid colon 105, the sigmoid colon 105 moves backward (in the direction of the anus) accompanied with the insertion of the colonoscope so as not to be extended excessively. And, owing to the insertion of the colonoscope and the motion of the sigmoid colon 105, a distance between the distal end of the descending colon 107 and the distal end of the colonoscope becomes short and therefore the sigmoid colon 105 and the descending colon 107 align substantially straight. And, when the distal end of the insertion tube 9 enters the transverse colon 109, the transverse colon 109 moves toward the splenic flexure 108 and the distal end of the insertion tube comes close to the hepatic flexure 110. As the result, the angle of the hepatic angle 110 becomes larger so that the colonoscope can easily pass through the hepatic flexure 117.

On the above described insertion process, a portion of the flexible section 15 provided with the endless belts 17 arranged on the outer wall of the flexible section 15 (that is, a portion of the flexible section 15 provided with the guide hooks 39, in other ward, a portion which provides a propelling force of the colonoscope) may be only a portion of the colonoscope which passes through the free segments (the sigmold colon 105 and the transverse colon 109) The sigmoid colon 105 is about 45 cm in length and the transverse colon 109 is about 50 cm in length. When the distal end of the colonoscope is inserted into the descending colon 107 for 10 cm, the distal end of the colonoscope should be inserted into the descending colon 107 from the sigmoid colon 105 completely. When the distal end of the colonoscope is inserted into the ascending colon 111 for 10 cm, the distal end of the colonoscope should be inserted into the ascending colon 111 from the transverse colon 109 via the hepatic flexure 110 completely.

As described above, when a length of a portion of the flexible section provided with the guide hooks 39 (an effective driving length of the endless belt) is set to about 60 cm which is a sum of a length of the transverse colon of a free segment and an allowance of 10 cm, it becomes possible to give sufficient self-propelling force to the colonoscope.

Then, the belt driving unit 5 and the casing 70 in which the belt driving unit 5 is stored will be described referring to FIG. 3.

The belt driving unit 5 is provided with three of gear assembly 50 (one of them is shown in FIG. 3), on which each of the three endless belts 17 is wound, and a motor 55 which drives the gear assemblies 50. The gear assemblies 50 are arranged at a regular angle (60°, in this example) around an output axis 55a of the motor 55.

Each of the gear assemblies 50 is provided with the driving roller 51 around which the endless belt 17 is wound, as shown in FIG. 3. The driving roller 51 has a pulley 51b on which the endless belt 17 is wound and a bevel gear 43a connected to the same shaft as the pulley 51b. On the circumferential surface of the pulley 51b, as shown in FIG. 5B, a concave groove is formed, in which the pinion gear teeth 51c are fixed, which engage with the rack gear teeth 18b of the endless belt 17.

To the output axis 55a of the motor 55, a large spur gear 59 is connected. A shown in FIG. 3, each of the gear assemblies 50 is further provided with a small spur gear 54 engaged with the larger spur gear 59 and a bevel gear 53 connected to a shaft 54a of the small spur gear 54 and engaged with the bevel gear 51a of the driving roller 51.

Consequently, when the motor shaft 55a revolves by driving the motor 55, the bevel gear 51a will revolve, via the large spur gear 59, the spur gear 54, and the bevel gear 53, and therefore the pulley 51b will also revolve and the endless belts 17 will travel.

The driving unit casing 70 has a cylindrical shape larger in diameter than the insertion tube 9, as shown in FIG. 1. As shown in FIGS. 1 and 3, the casing 70 is provided with a motor storage section 71 in which the motor 55 is stored; a gear assembly storage section 72 in which the gear assemblies 50 are stored; and a guide pipe hole section 73 at which the guide pipe holes 73a and 73b are formed, which are arranged in order away from the insertion tube 9.

As shown in FIG. 3, the motor storage section 71 is liquid-tightly separated from the gear assembly storage section 72. The output axis 55a of the motor 55 protrudes into the gear assembly storage section 72 via a seal.

The gear assembly storage section 72 is openable and closeable by a lid 75 as shown in FIG. 1. When the lid 75 is opened, the gears 51, 53 and 54 constituting the gear assembly 50 are exposed so as to be cleaned easily. And, it makes possible to attach or detach the gears, facilitating exchange of the gear. And, it makes also possible to clean the driving unit easily so that it become unnecessary to remove the endless belt after the colonoscopic examination.

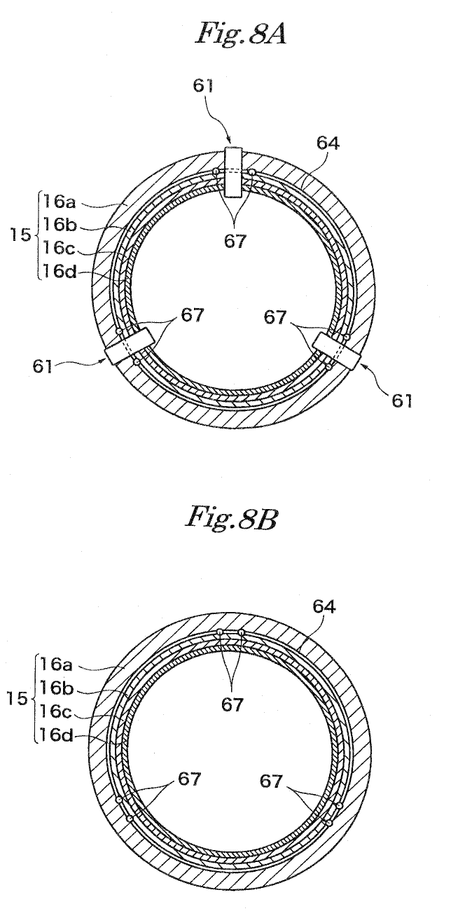

When the motor 55 is driven to rotate the pulley 51b counterclockwise in the figure, the exterior portion 17a of the endless belt 17 engaged with the pulley 51b will travel to the left direction in the figure. When the exterior portion 17a of the endless belt 17 is in contact with the inner wall of the colon, the insertion tube 9 will be propelled forward to the right direction in FIG. 8 by friction between the endless belts 17 and the inner wall of the colon. When the insertion tube 9 is propelled in the direction opposite to the insertion direction, the motor 55 is driven to rotate clockwise.

Next, another means for supporting the pulley 61 at the distal end of the circulating path of the endless belt will be described.

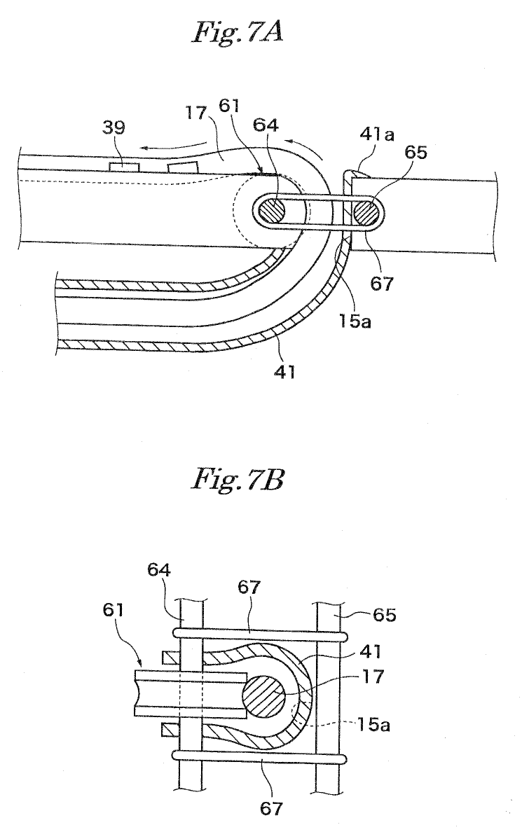

FIG. 7 are drawings showing a portion around the guide hole of the insertion tube 9, FIG. 7A is a vertical section drawing and FIG. 7B is a plan drawing.

FIG. 8 are transverse section drawings showing a portion around the guide hole of the insertion tube.

In the above embodiment, the rotating axis 63 of each of the pulleys 61 is supported by the guide pipe. In this embodiment, all of the pulleys 61 are rotatably supported by an axis belt 64 in exchange for the rotating axis.

The flexible section 15 is a flexible tube having a four layered structure comprising a coating layer 16a, a resin layer 16b, a mesh layer 16c and an inner and outer flex layer 16d from the outer surface in order, as shown in FIG. 8. The axis belt 64 is circumferentially arranged between the coating layer 16a and the resin layer 16b, as shown in FIG. 8A. The axis belt 64 supports each of the pulleys 61 rotatably.

Furthermore, in this embodiment, restriction means for keeping the axis belt 64 of the pulley 61 from moving toward the operation unit (driving unit, the left side of FIG. 7A) is provided. The restriction means comprises a restriction belt (pulley restriction means) 65 arranged in the flexible section 15 opposite the axis belt 64 behind the guide hole 15a and a coupling belt (coupling means) 67 coupling the restriction belt 65 and the axis belt 64.

The restriction belt 65 is circumferentially arranged between the coating layer 16a and the resin layer 16b as with the axis belt 64. The coupling belt 67 couples the axis belt 64 and the restriction belt 65 at the both sides of each of the pulleys 61. The coupling belt 67 keeps the axis belt 64 from moving toward the driving unit. As described above, since the endless belt 17 turns at the distal end of the circulating path (the guide hole 15a) at approximately 180°, the pulley 61 is subjected to force toward the driving unit and thus is pulled toward the same direction. So, by keeping the axis belt 64 from transferring toward the driving unit with the coupling belt 67, the endless belt 17 can turn smoothly.

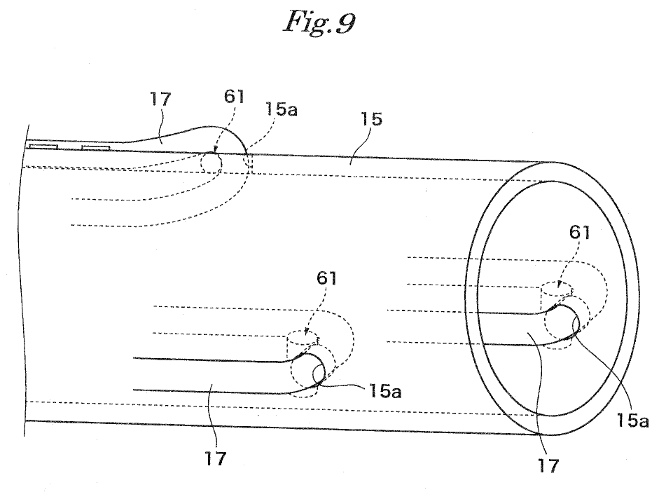

FIG. 9 is a drawing schematically showing a portion around the guide hole of the insertion tube of the self-propelled colonoscope according to another embodiment of the present invention.

As shown in FIG. 9, the guide holes 15a are formed at separate positions in the length direction of the flexible section 15. As with the above embodiment, the pulley 61 is attached on the side wall of the guide hole 15a on the side of the driving unit (operation unit).

If the guide holes 15a are formed at the same position in the length direction of the flexible section 15 and the pulley 61 is attached on the side wall of the guide hole 15a on the side of the driving unit, since the endless belts 17 get out though the guide holes 15a at the position, the diameter of the flexible section 15 becomes large partially at the position by the diameter of the endless belt 17. This increases resistance at the insertion. Accordingly, positions of the pulleys 61, that is positions of the guide holes 15a, are arranged at separate positions in the length direction of the flexible section 15. This disperses the diameter increased portions in the length direction of the flexible section 15, causing smoother insertion of the flexible section.

1-9. (canceled)

10. A self-propelled colonoscope comprising:
a tubular flexible insertion portion to be inserted into a colon;
an endless belt arranged along a circulating path on the inner wall and the outer wall of the flexible insertion portion;
a driving mechanism to drive the endless belt;
guide hooks arranged along the circulating path on the outer wall of the flexible insertion portion; and
a guide pipe extending along the circulating path on the inner wall of the flexible insertion portion and guiding the endless belt,
wherein at least a part of said guide hook is embedded in the outer surface of said flexible insertion portion.

11. The self-propelled colonoscope according to claim 10,
wherein said flexible insertion portion is compatible with an upper gastrointestinal endoscope in structure and size, except for said driving mechanism.

12. A self-propelled colonoscope comprising:
a tubular flexible insertion portion to be inserted into a colon;
an endless belt arranged along a circulating path on the inner wall and the outer wall of the flexible insertion portion;
a driving mechanism to drive the endless belt;
guide hooks arranged along the circulating path on the outer wall of the flexible insertion portion; and
a guide pipe extending along the circulating path on the inner wall of the flexible insertion portion and guiding the endless belt,
wherein a pulley is arranged at the distal end of the circulating path of said endless belt.

13. The self-propelled colonoscope according to claim 10,
wherein a pulley is arranged at the distal end of the circulating path of said endless belt.

14. The self-propelled colonoscope according to claim 12,
wherein a rotating axis for supporting said pulley rotatably is supported to said guide pipe.

15. The self-propelled colonoscope according to claim 12,
wherein said pulley is arranged on the side wall of said guide hole, at which said guide pipe is opened to the outer surface of said flexible insertion portion, on the side of said driving mechanism,
further comprising:
pulley restriction means arranged at a distal side of said guide hole and opposite to said pulley; and
coupling means for coupling said pulley restriction means and the rotating axis of said pulley,
wherein said coupling means restrains said pulley to said pulley restriction means to keep it from being pulled toward said driving unit.

16. The self-propelled colonoscope according to claim 12,
wherein the circulating path on the outer wall of the flexible insertion portion at the distal end portion of the flexible insertion portion is 10 to 60 cm in length.

17. The self-propelled colonoscope according to claim 12,
wherein said guide pipe is made of an ultralow friction material.

18. The self-propelled colonoscope according to claim 12,
wherein said pulleys are arranged at separate positions in the length direction of said flexible insertion portion.

19. The self-propelled colonoscope according to claim 11,
wherein a pulley is arranged at the distal end of the circulating path of said endless belt.

20. The self-propelled colonoscope according to claim 13,
wherein a rotating axis for supporting said pulley rotatably is supported to said guide pipe.

21. The self-propelled colonoscope according to claim 13,
wherein said pulley is arranged on the side wall of said guide hole, at which said guide pipe is opened to the outer surface of said flexible insertion portion, on the side of said driving mechanism,
further comprising:
pulley restriction means arranged at a distal side of said guide hole and opposite to said pulley; and
coupling means for coupling said pulley restriction means and the rotating axis of said pulley,
wherein said coupling means restrains said pulley to said pulley restriction means to keep it from being pulled toward said driving unit.

22. The self-propelled colonoscope according to claim 14,
wherein said pulley is arranged on the side wall of said guide hole, at which said guide pipe is opened to the outer surface of said flexible insertion portion, on the side of said driving mechanism,
further comprising:
pulley restriction means arranged at a distal side of said guide hole and opposite to said pulley; and
coupling means for coupling said pulley restriction means and the rotating axis of said pulley,
wherein said coupling means restrains said pulley to said pulley restriction means to keep it from being pulled toward said driving unit.

23. The self-propelled colonoscope according to claim 13,
wherein the circulating path on the outer wall of the flexible insertion portion at the distal end portion of the flexible insertion portion is 10 to 60 cm in length.

24. The self-propelled colonoscope according to claim 14,
wherein the circulating path on the outer wall of the flexible insertion portion at the distal end portion of the flexible insertion portion is 10 to 60 cm in length.

25. The self-propelled colonoscope according to claim 15,
wherein the circulating path on the outer wall of the flexible insertion portion at the distal end portion of the flexible insertion portion is 10 to 60 cm in length.

26. The self-propelled colonoscope according to claim 13,
wherein said guide pipe is made of an ultralow friction material.

27. The self-propelled colonoscope according to claim 14,
wherein said guide pipe is made of an ultralow friction material.

28. The self-propelled colonoscope according to claim 15,
wherein said guide pipe is made of an ultralow friction material.

29. The self-propelled colonoscope according to claim 16,
wherein said guide pipe is made of an ultralow friction material.
